(19) United States
(12) Patent Application Publication
(10) Pub. No.: US 2019/0069845 A1
(43) Pub. Date: Mar. 7, 2019

(54) SMART EXCREMENT IDENTIFYING LEAK PROTECTING REPLACEMENT REMINDING DIAPER

(71) Applicant: Kei Ting WONG, Hong Kong (HK)

(72) Inventor: Kei Ting WONG, Hong Kong (HK)

(21) Appl. No.: 15/721,828

(22) Filed: Sep. 30, 2017

(30) Foreign Application Priority Data
Sep. 6, 2017 (CN) 201710795900.3

(51) Int. Cl. A61B 5/00; A61F 13/42

(52) U.S. Cl. CPC A61B 5/6808; A61B 5/207; A61F 13/42

(57) ABSTRACT

The present invention relates to a smart excrement identifying leak protecting replacement reminding diaper, made up of a front waist wrap, a back waist wrap and a crotch disposed between the front waist wrap and the back waist wrap, a reminder device is disposed at an exterior of the diaper; double layer frills are disposed at two sides of the crotch extending axially, the crotch comprises a liquid permeable topsheet, a flow guide layer, an absorbent layer and a liquid impermeable backsheet extended to the double layer frill disposed from top to bottom in order; the liquid impermeable backsheet is made of air permable liquid impermeable polytetrafluoroethylene film; a protrusion wrapping around the liquid permeable topsheet is disposed at two sides of the flow guide layer, a number of liquid permeable holes is disposed on the flow guide layer. The diaper of the present invention has a reasonable structure, can identify the excrement in the diaper, so as to remind whether replacement is needed, preventing early replacement of diapers causing waste and late replacement of diapers affecting skin health.

### FIELD OF INVENTION

The present invention relates to the field of smart diapers, in particular to a smart excrement identifying leak protecting replacement reminding diaper.

### BACKGROUND OF INVENTION

A diaper is a piece of cloth wrapping around a body of a baby or placed on a baby bed for receiving urine. Diaper always stays with a baby until he can control his excretion, much like his second layer of skin. Following technological advancements and improvement of living standards, there are more and more brands of diapers, including all kinds of smart replacement reminding diapers. However, such diapers will buzz as soon as urine appears, prematurely replacing the diaper causes waste of diapers, but late replacement will cause skin health problems.

### SUMMARY OF INVENTION

The technical problem to be solved by the present invention is: to overcome the aforementioned problem, a smart excrement identifying leak protecting replacement reminding diaper is provided. The diaper has a reasonable structure, can identify the excrement in the diaper, so as to remind whether replacement is needed, preventing early replacement of diapers causing waste and late replacement of diapers affecting skin health.

The technical solution used in the present invention to solve the technical problem is: a smart excrement identifying leak protecting replacement reminding diaper, made up of a front waist wrap, a back waist wrap and a crotch disposed between the front waist wrap and the back waist wrap, a reminder device is disposed at an exterior of the diaper.

Double layer frills are disposed at two sides of the crotch extending axially, the crotch comprises a liquid permeable topsheet, a flow guide layer, an absorbent layer and a liquid impermeable backsheet extended to the double layer frill disposed from top to bottom in order; the liquid impermeable backsheet is made of air permable liquid impermeable polytetrafluoroethylene film; a protrusion wrapping around the liquid permeable topsheet is disposed at two sides of the flow guide layer, a number of liquid permeable holes is disposed on the flow guide layer.

The inside of the double layer frill is sewn into one piece with the crotch, elastic bands are disposed at a central part and along an outer edge of the double layer frill, a number of arced guide pieces concave towards outside is disposed inside the side frill of the double layer frill extending axially.

The replacement reminder device comprises a controller board attached to the front waist wrap, control circuitry disposed inside the controller board, a battery and a detection probe; the detection probe is installed at an upper part of the outside of the crotch, two untouched conduction probes are disposed at an inside surface of the detection probe for detecting urine, a photovoltaic sensor is disposed at a central part of the detection probe for detecting faeces.

The photovoltaic sensor comprises a light source disposed on the detection probe, a light sensor and a light reflecting shield for reflecting the light source onto the light sensor; the light reflecting shield is in an elliptical shape; an interference preventing light guiding shield is disposed along the exterior surface of the light source; the conduction probes are electrically connected to the control circuitry through wires; a fly is disposed at an outside of the liquid impermeable backsheet for installing the detection probe, elastic cloth is disposed on the fly for fixing the detection probe in place.

In a preferred embodiment, a Bluetooth module and a buzzer is disposed inside the control circuitry for connecting to external communication devices.

In a preferred embodiment, magic tape is disposed at two ends of the front waist wrap and the back waist wrap that attaches together.

In a preferred embodiment, the absorbent layer is made up of hydrophilic nonwoven fabric.

In a preferred embodiment, the liquid permeable topsheet is made up of hydrophilic nonwoven fabric at a center part and hydrophobic nonwoven fabric at the two sides.

In a preferred embodiment, the flow guide layer is made of polytetrafluoroethylene.

In a preferred embodiment, the arced guide piece is made of plastic.

In a preferred embodiment, the light reflecting shield is made of organic glass or transparent plastic board.

The advantage of the present invention is: the smart excrement identifying leak protecting replacement reminding diaper, when in use of the present invention has double layer frill, can better protect against leaks; the replacement reminder device can identify whether the excrement in the diaper is faeces, when excrement appears, light reflected by the light reflector changes, control circuitry sounds a buzz; when urine fully fills the diaper, urine permeates to the bottom of the diaper, contacting the two conduction probes together and buzzes, so as to remind for replacement, can prevent early replacement of diapers causing waste and late replacement of diapers affecting skin health.

### BRIEF DESCRIPTION OF DRAWINGS

The present invention is further explained below with the accompanying drawings and embodiments.

FIG. 1 is an overall structural schematic diagram of a smart excrement identifying leak protecting replacement reminding diaper according to the present invention.

FIG. 2 is a central cross sectional diagram of a smart excrement identifying leak protecting replacement reminding diaper.

FIG. 3 is a structural schematic diagram of a photovoltaic sensor of a smart excrement identifying leak protecting replacement reminding diaper.

FIG. 4 is a structural schematic diagram of a circuitry connection of a smart excrement identifying leak protecting replacement reminding diaper.

Reference numbers in the drawings: 1 front waist wrap, 11 magic tape, 2 back waist wrap, 3 crotch, 31 liquid permeable topsheet, 32 flow guiding layer, 321 protrusion, 322 liquid permeable hole, 33 absorbent layer, 34 liquid impermeable backsheet, 341 fly, 342 elastic cloth, 4 double layer frill, 41 elastic band, 42 arced guide piece, 5 controller, 50 control circuitry, 51 battery, 52 detection probe, 53 conduction probe, 54 Bluetooth module, 55 buzzer, 6 light source, 61 light sensor, 62 interference preventing light guiding shield, 53 light reflecting shield.

### DETAILED DESCRIPTION OF INVENTION

The present invention is further explained below along with the accompanying drawings. The drawings are all simplified schematic diagrams which only illustrates the basic structure of the present invention, as such only the components material to the present invention are shown.

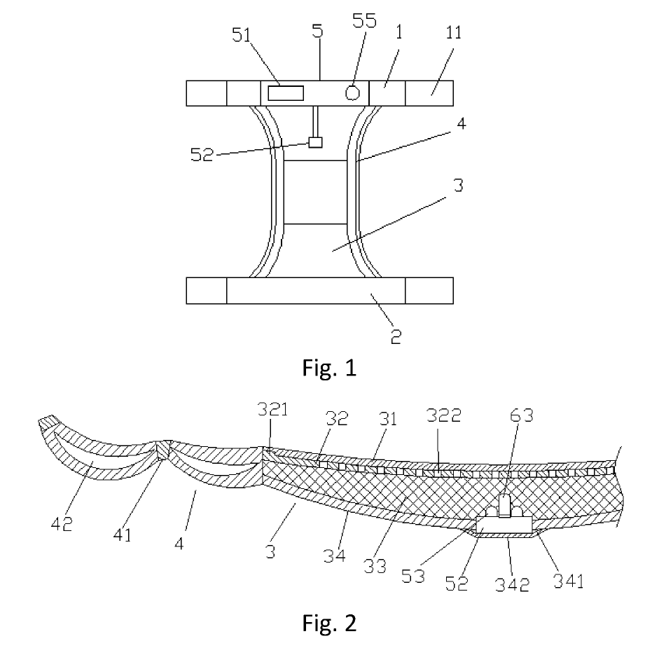

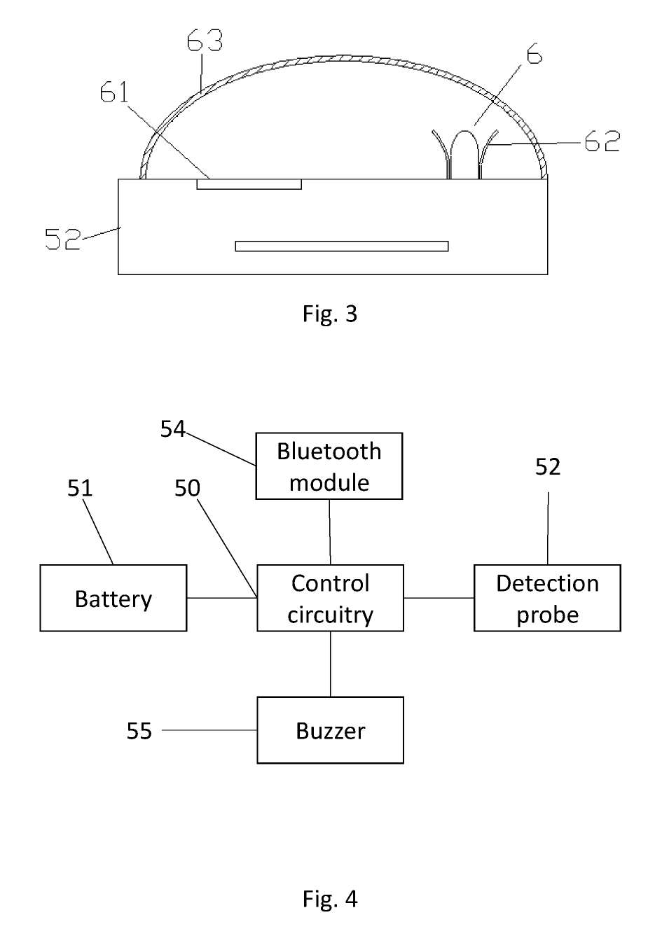

A smart excrement identifying leak protecting replacement reminding diaper is shown in FIGS. 1, 2, 3 and 4. The diaper is made up of a front waist wrap 1, a back waist wrap 2 and a crotch 3 disposed between the front waist wrap 1 and the back waist wrap 2, a reminder device is disposed at an exterior of the diaper; double layer frills 4 are disposed at two sides of the crotch extending axially, the crotch 3 comprises a liquid permeable topsheet 31, a flow guide layer 32, an absorbent layer 33 and a liquid impermeable backsheet 34 extended to the double layer frill 4 disposed from top to bottom in order; the liquid impermeable backsheet 34 is made of air permable liquid impermeable polytetrafluoroethylene film; a protrusion 321 wrapping around the liquid permeable topsheet 31 is disposed at two sides of the flow guide layer 32, a number of liquid permeable holes 322 is disposed on the flow guide layer 32;

The inside of the double layer frill 4 is sewn into one piece with the crotch 3, elastic bands 41 are disposed at a central part and along an outer edge of the double layer frill 4, a number of arced guide pieces 42 concave towards outside is disposed inside the side frill of the double layer frill 4 extending axially.

The replacement reminder device comprises a controller board 5 attached to the front waist wrap 1, control circuitry 50 disposed inside the controller board 5, a battery 51 and a detection probe 52; the detection probe 52 is installed at an upper part of the outside of the crotch 3, two untouched conduction probes 53 are disposed at an inside surface of the detection probe 52 for detecting urine, a photovoltaic sensor is disposed at a central part of the detection probe 52 for detecting faeces.

The photovoltaic sensor comprises a light source 6 disposed on the detection probe 52, a light sensor 61 and a light reflecting shield 63 for reflecting the light source 6 onto the light sensor 61; the light reflecting shield 63 is in an elliptical shape; an interference preventing light guiding shield 62 is disposed along the exterior surface of the light source 6; the conduction probes 53 are electrically connected to the control circuitry 50 through wires; a fly 341 is disposed at an outside of the liquid impermeable backsheet 34 for installing the detection probe 52, elastic cloth 342 is disposed on the fly for fixing the detection probe 52 in place.

A Bluetooth module 54 and a buzzer 55 is disposed inside the control circuitry 50 for connecting to external communication devices. Magic tape 11 is disposed at two ends of the front waist wrap 1 and the back waist wrap 2 that attaches together. The absorbent layer 33 is made up of hydrophilic nonwoven fabric. The liquid permeable topsheet 31 is made up of hydrophilic nonwoven fabric at a center part and hydrophobic nonwoven fabric at the two sides. The flow guide layer 32 is made of polytetrafluoroethylene. The arced guide piece 42 is made of plastic. The light reflecting shield 63 is made of organic glass or transparent plastic board.

The smart excrement identifying leak protecting replacement reminding diaper, when in use of the present invention has double layer frill, can better protect against leaks; the replacement reminder device can identify whether the excrement in the diaper is faeces, when excrement appears, light reflected by the light reflector changes, control circuitry sounds a buzz; when urine fully fills the diaper, urine permeates to the bottom of the diaper, contacting the two conduction probes together and buzzes, so as to remind for replacement, can prevent early replacement of diapers causing waste and late replacement of diapers affecting skin health.

As revealed by the preferred embodiments of the present invention, based on the aforementioned description, a person skilled in the art can make various modifications and adjustments without departing from the technical concept of the present invention. The technical scope of the present invention is not limited to the specification but is defined by the scope of the claims.

What is claimed is:

1. A smart excrement identifying leak protecting replacement reminding diaper, made up of a front waist wrap, a back waist wrap and a crotch disposed between the front waist wrap and the back waist wrap, a reminder device is disposed at an exterior of the diaper;
double layer frills are disposed at two sides of the crotch extending axially, the crotch comprises a liquid permeable topsheet, a flow guide layer, an absorbent layer and a liquid impermeable backsheet extended to the double layer frill disposed from top to bottom in order;
the liquid impermeable backsheet is made of air permable liquid impermeable polytetrafluoroethylene film; a protrusion wrapping around the liquid permeable topsheet is disposed at two sides of the flow guide layer, a number of liquid permeable holes is disposed on the flow guide layer;
the inside of the double layer frill is sewn into one piece with the crotch, elastic bands are disposed at a central part and along an outer edge of the double layer frill, a number of arced guide pieces concave towards outside is disposed inside the side frill of the double layer frill extending axially;
the replacement reminder device comprises a controller board attached to the front waist wrap, control circuitry disposed inside the controller board, a battery and a detection probe; the detection probe is installed at an upper part of the outside of the crotch, two untouched conduction probes are disposed at an inside surface of the detection probe for detecting urine, a photovoltaic sensor is disposed at a central part of the detection probe for detecting faeces;
the photovoltaic sensor comprises a light source disposed on the detection probe, a light sensor and a light reflecting shield for reflecting the light source onto the light sensor; the light reflecting shield is in an elliptical shape; an interference preventing light guiding shield is disposed along the exterior surface of the light source; the conduction probes are electrically connected to the control circuitry through wires; a fly is disposed at an outside of the liquid impermeable backsheet for installing the detection probe, elastic cloth is disposed on the fly for fixing the detection probe in place.

2. The smart excrement identifying leak protecting replacement reminding diaper according to claim 1, characterized in that: a bluetooth module and a buzzer is disposed inside the control circuitry for connecting to external communication devices.

3. The smart excrement identifying leak protecting replacement reminding diaper according to claim 1, characterized in that: magic tape is disposed at two ends of the front waist wrap and the back waist wrap that attaches together.

4. The smart excrement identifying leak protecting replacement reminding diaper according to claim 1, characterized in that: the absorbent layer is made up of hydrophilic nonwoven fabric.

5. The smart excrement identifying leak protecting replacement reminding diaper according to claim 1, characterized in that: the liquid permeable topsheet is made up of hydrophilic nonwoven fabric at a center part and hydrophobic nonwoven fabric at the two sides.

6. The smart excrement identifying leak protecting replacement reminding diaper according to claim 1, characterized in that: the flow guide layer is made of polytetrafluoroethylene.

7. The smart excrement identifying leak protecting replacement reminding diaper according to claim 1, characterized in that: the arced guide piece is made of plastic.

8. The smart excrement identifying leak protecting replacement reminding diaper according to claim 1, characterized in that: the light reflecting shield is made of organic glass or transparent plastic board.
